(19) United States
(12) Patent Application Publication
(10) Pub. No.: US 2005/0037089 A1
(43) Pub. Date: Feb. 17, 2005

(54) Method for promoting bone growth

(76) Inventor: Jill Marie Jobbins, Freehold, NJ (US)

Correspondence Address: KEVIN E. MC VEIGH;RHODIA INC., 259 PROSPECT PLAINS ROAD, CRANBURY, NJ, 08512, US

(21) Appl. No.: 10/909,894

(22) Filed: Aug. 2, 2004

Related U.S. Application Data
(60) Provisional application No. 60/492,882, filed on Aug. 6, 2003.

(51) Int. Cl. A61K 38/29; A61K 31/66; A61K 33/42

(52) U.S. Cl. 424/602; 514/12; 514/102

(57) ABSTRACT

A method for treating a patient to promote bone growth, comprising: administering to the patient a therapeutic amount of a bone remodeling agent effective to stimulate bone growth, and a dietary supplement comprising calcium and phosphorus, in a therapeutic amount effective to prevent a deficiency in the amount of calcium or phosphorus available for bone growth.

### FIELD OF THE INVENTION

This invention relates to a method for promoting bone growth.

### BACKGROUND OF THE INVENTION

Osteoporosis is a condition characterized by progressive thinning of bones, including reduced bone mineral density and reduced bone quality, that may lead to increased risk of bone, particularly spine, hip and wrist, fractures. Osteoporosis is a global problem that affects more than 150 million women worldwide.

It is widely believed that a chronic shortage of dietary calcium is one factor leading to osteoporosis, see Osteoporosis, Cause, Treatment, Prevention, U.S. Dept. of Health and Human Services, Public Health Service National Institutes of Health, Maryland 1987. Calcium dietary supplements appear to be of value in helping to prevent osteoporosis.

A number of therapies are available for prevention or treatment of osteoporosis. Various bone remodeling agents, for example, hormones such as estrogen and progesterone, bisphosphonates such as alendronate, risedronate, editronate, tiludronate, and clodronate, selective estrogen receptor modulators such as raloxifene, and other agents such as calcitonin and calcitriol, typically administered in combination with dietary calcium supplementation, have each been shown to improve bone mineral density, see, for example, Watts, Nelson B., Therapies to Improve Bone Mineral Density and Reduce Risk of Fracture, Journal of Reproductive Medicine, 0024-7758/02/4701-0082 and Kleerkoper, Micheal, et. al., Comparative Safety of Bone Remodeling Agents with a Focus on Osteoporosis Therapies, Journal of Clinical Pharmacology, 2001:41:239-250. More recently, subcutaneous administration of the N-terminal fragment 1-34 of parathyroid hormone (referred to as hPTH(1-34) or teriparatide) has been shown to increase bone formation in patients with osteoporosis, see Neer, Robert M., et. al., Effect of Parathyroid Hormone (1-34) on Fractures and Bone Mineral Density in Postmenopausal Women with Osteoporosis, The New England Journal Of Medicine, Volume 344, No. 19, pp 1434-1441. Parathyroid hormone (PTH) is an 84-amino acid polypeptide that regulates extracellular calcium homeostasis by facilitating calcium absorbtion. FORTEO® (teriparatide [rDNA origin] injection, Eli Lilly and Company) has been approved by the U.S. Federal Drug Administration for the treatment of osteoporosis patients, including men with primary or hypogonadal osteoporosis as well as postmenopausal women and, who are at high risk for having a bone fracture.

It also has been suggested that other minerals in addition to calcium, such as copper, magnesium and zinc, and certain vitamins, such as vitamin D, play important roles in bone formation and metabolism, see, for example, Strause, L., et. al., “The Role of Trace Elements in Bone Metabolism”, Nutritional Aspects of Osteoporosis, New York, Raven Press, p. 223-233, 1992.

### SUMMARY OF THE INVENTION

In a first aspect, the present invention is directed to a method for treating a patient to promote bone growth, comprising:
administering to the patient:
   a bone remodeling agent, in an amount effective to stimulate bone growth, and
   a dietary supplement comprising calcium and phosphorus, in an amount effective to prevent a deficiency in the amount of calcium or phosphorus available for bone growth.

### DETAILED DESCRIPTION OF INVENTION AND PREFERRED EMBODIMENTS

Compounds suitable for use as the bone remodeling agent component of the present invention are those that are administered in therapeutic dosages to human patients having a medical condition for which such administration is indicated in order to promote an increase in bone mass density and/or reduce risk of bone fracture. Suitable bone remodeling agents include hormones, such as estrogen, progesterone, parathyroid hormone, parathyroid-related hormone, bisphosphonates such as alendronate, risedronate, editronate, tiludronate, and clodronate, selective estrogen receptor modulators such as raloxifene, and other agents, such as calcitonin and calcitriol.

In one embodiment, the bone remodeling agent component of the present invention comprises one or more bone remodeling agent selected from hormones, bisphosphonates, selective estrogen receptor modulators, calcitonin, and calcitriol.

In one embodiment, the bone remodeling agent comprises one or snore of parathyroid hormone, a parathyroid hormone fragment, or human parathyroid related hormone.

Parathyroid hormone is a polypeptidic hormone that elevates calcium level by dissolving salts in bone and preventing their renal excretion. Human parathyroid-related hormone 1-34 (hPTH(1-34)) is the N-terminal fragment 1-34 of human parathyroid hormone. Studies have shown that the hPTH(1-34) has the same biological properties as the intact parathyroid hormone in stimulating bone formation.

The overall structure of hPTH(1-34) has been characterized as a slightly bent helix, with the bend located between residues 12 and 21 with a bend angle of 15° between the N-terminal helix (residues 3-11) and the C-terminal helix (residues 21-33), see Jin, Lei et. al. Crystal Structure of Human Parathyroid Hormone 1-34 at 0.9 Å Resolution, J. Biol. Chem. Vol. 275, No. 35, pp. 27238-27244.

In one embodiment, the amount of hormone effective to stimulate bone growth is from about 20 to about 40 micrograms (μg) of hPTH(1-34)) per day. In one embodiment, the effective amount of hPTH(1-34)) is administered subcutaneously. Daily subcutaneous administration of 20 μg or 40 μg of hPTH(1-34)) was found to increase bone density in postmenopausal women, see Neer et. al. above.

In one embodiment, the supplement composition of the present invention comprises from about 1 to about 4, more typically from about 1 to about 3, parts by weight (“pbw”) calcium per pbw phosphorus.

In one embodiment, the effective amount of calcium is from about 500 to about 2000, more typically from about 1000 to about 1800 milligrams (mg) calcium per day and the effective amount of phosphorus is from about 200 to about 1600, more typically from about 400 to about 1200 mg phosphorus per day.

In one embodiment, the calcium component of the supplement composition of the present invention comprises one or more of calcium chelates, such as for example, calcium proteinate, and calcium salts, such as, for example, calcium carbonate, calcium gluconate, calcium citrate, tricalcium phosphate, or dicalcium phosphate dihydrate or anhydrous dicalcium phosphate, calcium citrate maleate.

In one embodiment, the phosphorus component of the supplement composition of the present invention comprises one or more of phosphate salts, such as for example, tricalcium phosphate, dicalcium phosphate dihydrate, anhydrous dicalcium phosphate, sodium phosphate, magnesium phosphate, and potassium phosphate, and proteins, such as, for example, soy protein, and whey protein.

In one embodiment, the phosphorus component and a portion of the calcium component of the supplement composition of the present invention are supplied as a calcium phosphate salt, such as for example, tricalcium phosphate, dicalcium phosphate dihydrate, anhydrous dicalcium phosphate, monocalcium phosphate.

In one embodiment, the dietary supplement composition of the present invention comprises anhydrous dicalcium phosphate, tricalcium phosphate, and calcium carbonate.

In one embodiment, the supplement composition further comprises dietary supplemental amounts of vitamins or minerals other than calcium or phosphorus. In a highly preferred embodiment, the supplement composition comprises a dietary supplemental amount of one or more vitamins, such as for example, vitamin D, Vitamin B₆; folate and Vitamin B₁₂, phytoestrogens, such as for example, one or more isoflavones, one or more probiotics and prebiotics, such as for example lactobacillus acidophilus, inulin or other polysaccharides and one or more minerals other than calcium or phosphorus, such as for example, boron, copper, zinc, magnesium, manganese and zinc, that play a role in bone formation and/or bone metabolism. Alternatively, the supplement composition of the present composition may be included as a component of a multi-vitamin and mineral supplement composition.

The supplement composition of the present invention may, optionally, contain other ingredients generally recognized as safe for food additive use, including for example, preservatives, such as, for example, butylated hydroxytoluene, butylated hydroxyanisole, food grade emulsifiers, such as, for example, lecithin, propylene glycol esters, and pharmaceutically acceptable carriers and excipients, such as for example, binders, fillers, lubricants, dissolution aids.

The supplement composition of the present invention is made by combining calcium and phosphorus components, as well as any optional components, in the desired relative amounts and mixing the components according to known methods to produce a substantially homogeneous mixture.

The present supplement composition may be administered in any oral dosage form, including liquid dosage forms such as, for example, a suspension or slurry, and oral solid dosage forms such as, for example, a tablet, bulk powder, or soft chews. As used herein the term “tablet” refers generally to tablets, chewable tablets, caplets, capsules, including soft gelatin capsules, and lozenges, and the term “soft chews” refers to dosage forms wherein calcium and phosphorus components are provided in a soft, chewable candy base.

In one embodiment, the supplement composition of the present invention is administered in the form of a tablet. Tablets are made by methods known in the art and may further comprise suitable binders, lubricants, diluents, disintegrating agents, colorants, flavoring agents, flow-inducing agents, melting agents, which are known in the art. The oral solid dosage form may, optionally, have a film coating to protect the components of the supplement composition from one or more of moisture, oxygen and light or to mask any undesirable taste or appearance. Suitable coating agents include, for example, cellulose, hydroxypropylmethyl cellulose.

### EXAMPLE

A patient is administered a subcutaneous dosage of 20 μg of hPTH(1-34)) per day and an oral dosage of 2 nutritional supplement tablet(s), each comprising 600 mg calcium and 266 mg phosphorus per day.

1. A method for treating a patient to promote bone growth, comprising:
administering to the patient:
a bone remodeling agent, in an amount effective to stimulate bone growth, and
a dietary supplement comprising calcium and phosphorus, in an amount effective to prevent a deficiency in the amount of calcium or phosphorus available for bone growth.

2. The method of claim 1, wherein the bone remodeling agent comprises one or more bone remodeling agents selected from hormones, bisphosphonates, selective estrogen receptor modulators, calcitonin, and calcitriol.

3. The method of claim 1, wherein the bone remodeling agent comprises one or more of parathyroid hormone, a parathyroid hormone fragment, or human parathyroid related hormone.

4. The method of claim 1, wherein the bone remodeling agent comprises human parathyroid-related hormone 1-34.

5. The method of claim 4, wherein the amount of hormone effective to stimulate bone growth is from about 20 to about 40 micrograms of human parathyroid-related hormone 1-34 per day.

6. The method of claim 1, wherein the dietary supplement comprises from about 1 to about 4 parts by weight calcium per parts by weight phosphorus.

7. The method of claim 1, wherein the effective amount of calcium is from about 500 to about 2000 milligrams calcium per day and the effective amount of phosphorus is from about 200 to about 1600 milligrams phosphorus per day.

8. The method of claim 1, wherein dietary supplement comprises one or more compounds selected from calcium chelates and calcium salts.

9. The method of claim 1, wherein dietary supplement comprises one or more compounds selected from phosphate salts and proteins.

10. The method of claim 1, wherein the phosphorus component and a portion of the calcium component of the dietary supplement are supplied as a calcium phosphate salt.

11. The method of claim 1, wherein the dietary supplement comprises one or more compounds selected from tricalcium phosphate, dicalcium phosphate dihydrate, anhydrous dicalcium phosphate, and monocalcium phosphate.

12. The method of claim 11, wherein the dietary supplement further comprises one or more compounds selected from anhydrous dicalcium phosphate, tricalcium phosphate, and calcium carbonate.

13. The method of claim 11, wherein the dietary supplement further comprises dietary supplemental amounts of one or more compounds selected from vitamins and minerals other than calcium or phosphorus.
